(19) United States
(12) Patent Application Publication
(10) Pub. No.: US 2022/0362547 A1
(43) Pub. Date: Nov. 17, 2022

(54) Grounding skin care system

(71) Applicant: Jeremy Madvin, Westlake Village, CA (US)

(72) Inventor: Jeremy Madvin, Westlake Village, CA (US)

(21) Appl. No.: 17/321,508

(22) Filed: May 17, 2021

(51) Int. Cl. A61N 1/14

(52) U.S. Cl. CPC A61N 1/14; A61H 39/002

(57) ABSTRACT

The present invention discloses an enhanced combination of skin care system with grounding system which includes conductive skin care tropical which can be in the form of crème, gel or hydrogel patch and grounding mechanism to perform more refined and dedicated approach towards the affected area. As per an embodiment of the invention provides a method of treating a target tissue site method comprising selecting the tissue site based on area selected by user which can be a small or wide area; delivering energy to the tissue site at a first depth to achieve a first tissue effect using an energy delivery device; and remodeling at least a portion of tissue at the tissue site.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to a grounding skin care system and in particular to a grounding skin care system including a grounding mechanism and a conductive skin care topical including but not limited to in the form of crème, gel and hydrogel patch.

### Description of the Related Art

The largest living organ, the skin, defends the skin from the outside world by regulating temperature and fluid balance, keeping pathogenic organisms and chemicals out, and providing sun protection. The stratum corneum, the outmost layer of skin, is a selectively permeable, heterogeneous layer of the epidermis that protects against desiccation and environmental challenges while retaining skin integrity. Damage to this layer results in loss of skin integrity, and chances of infection increase.

The main goals of acute and long-term skincare are protecting and improving skin health and integrity. Skin integrity is a quality indicator, and it is generally agreed that preserving skin integrity is more cost-effective than wound treatment. Patients who are seriously and chronically ill and those who are immobile or have incontinence are at risk for various skin problems, including incontinence-associated dermatitis, pressure ulcers, intertriginous dermatitis, and skin drops. Age advancement can be considered an independent risk factor for developing skin problems due to ongoing changes in skin and associated soft tissue function and structure. Skincare routines are based on personal preferences, beliefs, and local care strategies instead of current best evidence-based practices.

The Federal Act on Food, Drugs and Cosmetics defines cosmetics as products of beauty and cleanliness (for instance, shampoos and lipstick). And dermatology involves medicines designed to diagnose, cure, mitigate, treat, or prevent disease or affect body structure and function (for example, sunscreens and acne creams); however, several other products are also present in this category, such as sunscreens moisturizing and shampoos anti-dandruff.

Traditionally, skincare differs from applied dermatology because it has an additional component, as well as fewer formal medical components. Skincare can be administrated by all tiers of medical and non-medical professionals such as aestheticians, CAM specialists' nurses and even untrained people with concerns about skin health. Skincare includes changes in individual behavior, environmental conditions, and working conditions. In America, skincare is a separate discipline, whereas it has been co-opted by dermatology in the U.K.

Skincare technology is diverse and, because of the functionality of many of the products, it differs from many cosmetic groups. There are beauty products and pharmacological products. On the other hand, many of the items are made to interact with and support the body's largest organ—the skin. Skincare involves several procedures that help maintain the skin's integrity, improve its appearance, and alleviate skin problems. They may include a balanced diet, avoiding unnecessary sun exposure, and using emollients appropriately. Cosmetics, exfoliation, botulinum toxin, laser resurfacing, fillers, microdermabrasion, retinol therapy, peels, and ultrasonic skin treatment are methods for improving skin appearance. Skincare is routine daily practices in many situations, including skin too dry or too moist, dermatitis prevention, and skin injury prevention.

Increased blood flow to your body revitalizes the skin. There are many treatments available to increase blood flow. Some people use cream with specific ingredients, while other use direct skin stimulation (ultrasound, massages, and lasers), chemical peels, and dermabrasion. While these therapies produce results, but they can have significant short- or long-term side effects. Many techniques that encourage the remodeling and synthesis of the dermal matrix and collagen synthesis require extensive post-treatment care and may cause complications, such as pain, infections, bleeding, erythema, oozing, burning, and scaring. On the other hand, grounding is a simple alternative to increasing localized blood flow, requires little or no effort and can easily be integrated into everyday life.

Grounding implies the connection of the human body to the regular and active electrical charge present in the Earth. It is a natural lifestyle exercise that systemically affects the body's fundamental bioelectrical function. Grounding may stabilize physiology remarkably, decreases pain and inflammation, increase the blood flow and venous/lymphatic flow back to the heart, enhance sleep, and decrease anxiety and stress, creating greater well-being. People also report that they look and feel younger and healthier after grounding. Many with pain experience less pain. Even morale has been reported to increase. Grounding is simple to perform and, especially for people with chronic health problems, often produces fast results.

Grounding has two basic types: 1) indoors, using economic Grounding devices (conductive sheets, mats, hand and body patches or bands) either during sleeping or working, and 2) outdoors, barefoot in contact with natural conductive surfaces such as grass, stone, dirt, sand, and gravel. Outdoor grounding is the most common and natural form of Grounding.

Due to modern human lifestyle, grounding restores a primeval electrical link to the Planet. Grounding seems to fix an “electron deficiency syndrome,” a neglected and possibly leading reason for different health circumstances. Grounding is a powerful method for preventing and rehabilitating health and healing. This technique can be used independently or in combination with clinical therapies to alleviate symptoms and eliminate the root causes of diseases and disorders that start with an imbalance in the different body bioelectric levels.

It is important that the medical community grasp that a body attached to the Earth is likely healthier than an ungrounded body. The body simply works more efficiently and under less cellular stress. Humans have been increasingly disconnected from the Planet. Obviously, now, we do not regularly sleep on the grass, do not regularly walk outside without shoes, and commonly wear insulating plastic sole shoes in place of conventional and conductive leather shoes. We work and live-in high-rise buildings, sometimes thousands of feet above the ground, and spend most of our time disconnected from the Earth.

Electrical imbalances, the buildup of harmful positive charged particles, and an unknown body deficiency of electrons can result from a lack of interaction with the Earth and susceptibility to malfunction, disease, and disorder. As is well known, Planet Earth's surface is electrically influenced by solar radiation, electrical encounters such as lightning bolts, and other dynamic forces of the atmosphere. However, it is not common knowledge that these phenomena give land masses and water bodies a continually refreshed supply of free electrons passing the Earth with a regular negative electrical charge. According to biological Grounding science, this same electrical charge, with its infinite electron supply and circadian frequencies, serves as a regulating and supporting purpose for both plant and animal kingdoms, akin to “electrical nutrition.” The findings back up the theory that Grounding allows many free electrons to move into the body, causing rapid physical changes, often instantaneous. Grounding preserves and retains an internal electrical condition that is normal. A few years ago, two Polish scholars and doctors, Sokal and Sokal, performed different experiments and resulted in a conclusion that the human body is a “universal controlling force in Nature”. Bioelectric, biochemical, and bioenergetic processes are highly regulated and tend to have a considerable controlling effect on chronic diseases and disorders.

Electro- and chemical reactions that impact the essential biological process in the human body can be triggered by external physical stimulation. Grounding therapy is also regarded in oriental medicine as induction of such reaction, allowing unstable physiological situations to return to normal physiologically balanced conditions. Grounding therapy has explained the theory of physiologic therapy in Western medicine as a cytological and endocrine nerve control process mechanism. Grounding results in brain responses; it may not be accurate for all reactions to occur only through the nervous system because the biological response to external stimulation through the nervous system is much faster than is possible. It indicates that biological methods other than the nervous system could be involved.

It has been stated that stimulation of the skin with grounding appears to produce an ion-electric-charge, which is transmitted via various biological pathways-blocking the ion-electric-charge results in an unbalanced negative and positive charge in an organ and hence pain and the generation of multiple diseases. Grounding moves the excessive or insufficient state of the ion-electric charge to allow good health to be restored. It suggests that grounding is intricately linked to the transfer of bioelectrical charges. When skincare is combined with simultaneous grounding, mass therapy provides an increased likelihood of positive effects with no potential consequences.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable.

It has to be noted that the current invention proposes an assembly with multiple advancements including a structure of grounding mechanism and a conductive skin care topical (in the form of crème, gel, hydrogel patch, etc.). The skin care can include other ingredients that aid in moisturizing, wrinkle reduction and so on.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a grounding skin care system including a conductive, medicated interface for simple, non-invasive and accessible techniques in single unit.

It is another objective of the invention to provide a conductive medicine which can be applied on any part of body for enhanced treatment.

It is also the objective of invention to provide a method of treating a target tissue site, the method comprising selecting the tissue site based on user's requirement; delivering energy to the tissue site at a first depth to achieve a first tissue effect using an energy delivery device.

It is another objective of the invention to provide a skin care system which being used is increasingly agreeable for the patient.

It is further the objective of the invention to provide a simple, non-invasive, and accessible techniques of skin care system whereby the lotion, crème, gel or other topical which includes ingredients (for moisturizing, wrinkle reduction, etc.) is conductive.

It is also the objective of the invention to provide a device of the type described which provides variety of relaxation techniques, exercises and stretches that assist to restore and recover problematic soft tissues in the body.

It is also the objective of the invention to provide a device which increases the results of a grounding wire which is connected to conductive skin care lotion, gel, crème, etc. to enhance the outcomes of the lotion, gel, crème, etc.

It is further the objective of the invention to restore issues associated with pain enhancing the effects of grounding through allowing for a pinpointed ion gateway.

It is also the objective of invention which is also effective in potentiating its biological and clinical benefits, while reducing costs at the same time.

A further object is to provide an assembly that is simple, rapidly adjusted and easy to use.

A still further object is to provide a skin care system that is economical in cost to manufacture.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 discloses the general working phenomenon as per preferred embodiments of the invention.

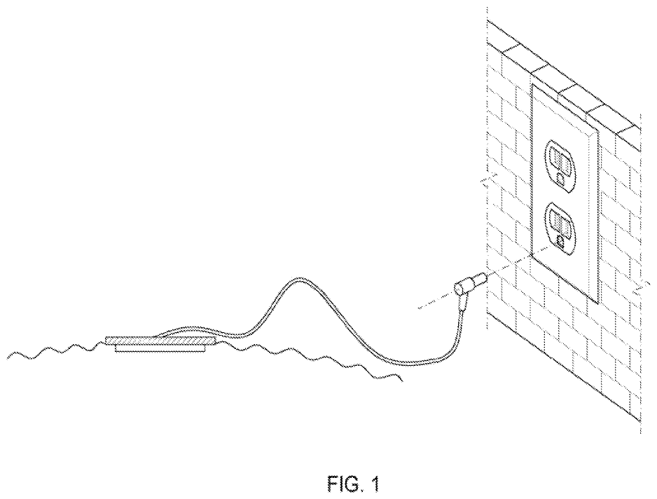

FIG. 2 discloses perspective use as per preferred embodiments of the invention.

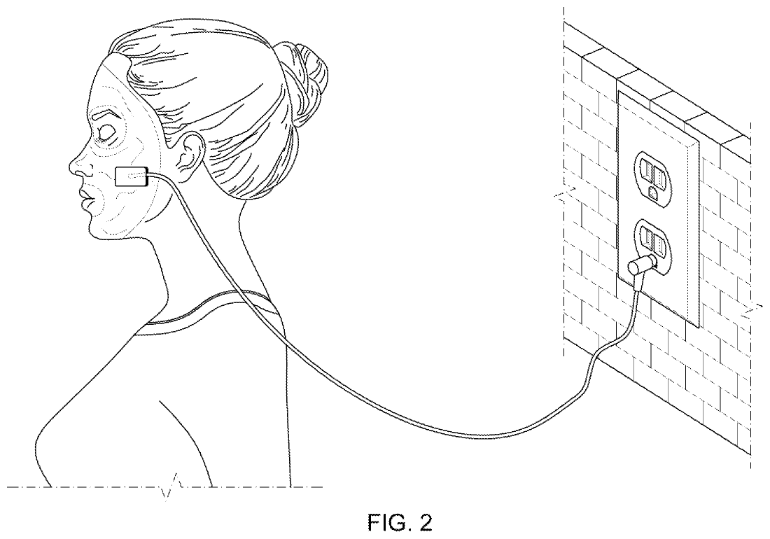

FIG. 3 discloses another view of perspective use as per preferred embodiments of the invention.

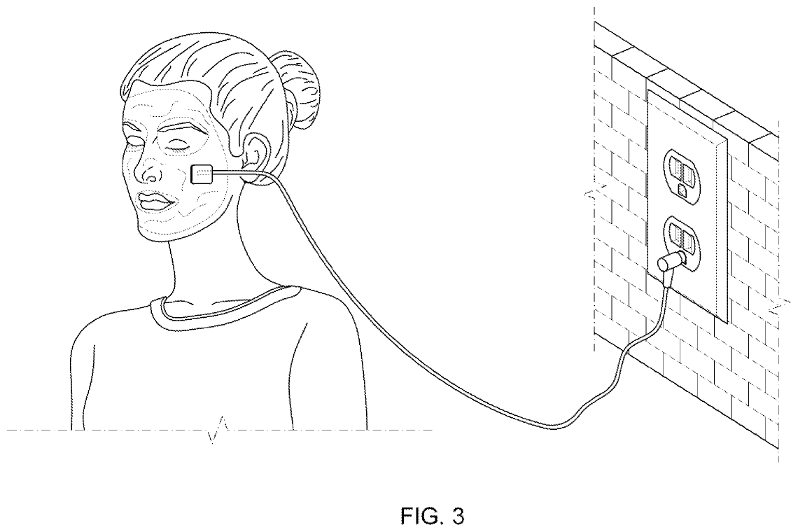

FIG. 4 discloses front view of user as the proposed invention is in use.

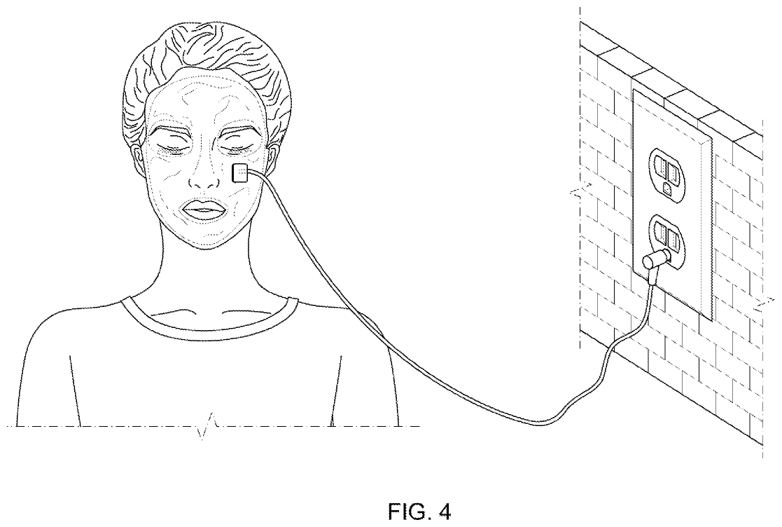

FIG. 5 discloses the Grounding prong, wire, mask and snapping button as per preferred embodiments of the invention.

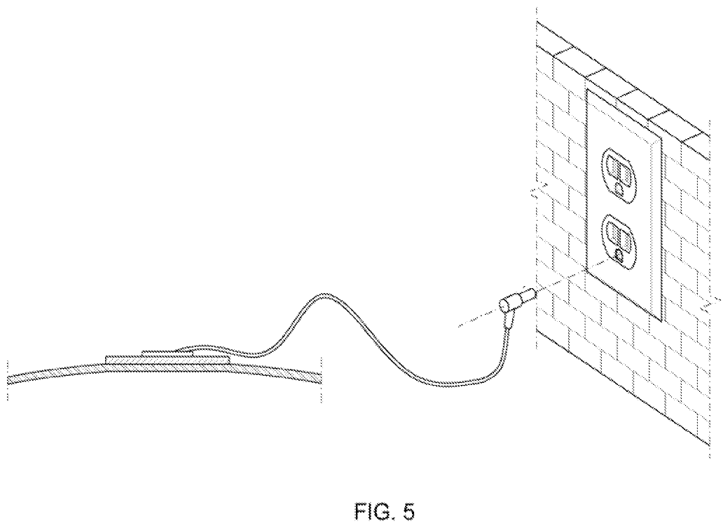

FIG. 6 discloses another view of perspective use as per preferred embodiments of the invention.

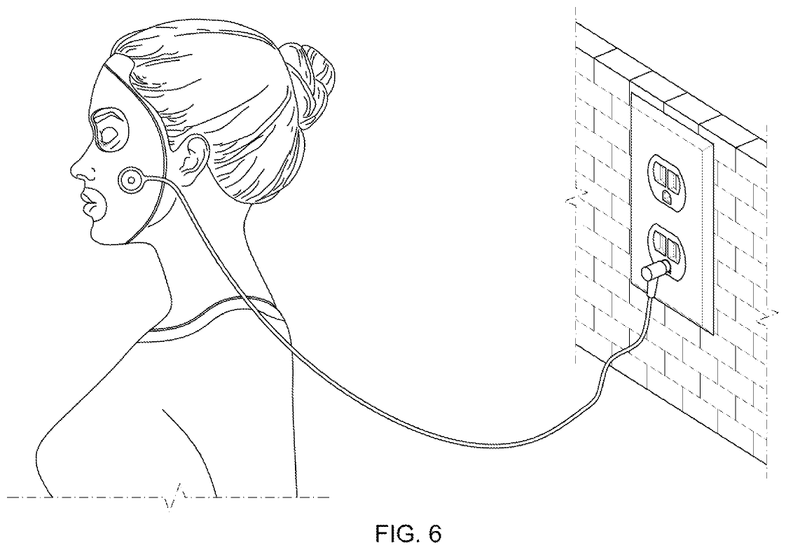

FIG. 7 discloses another view of perspective use as per preferred embodiments of the invention.

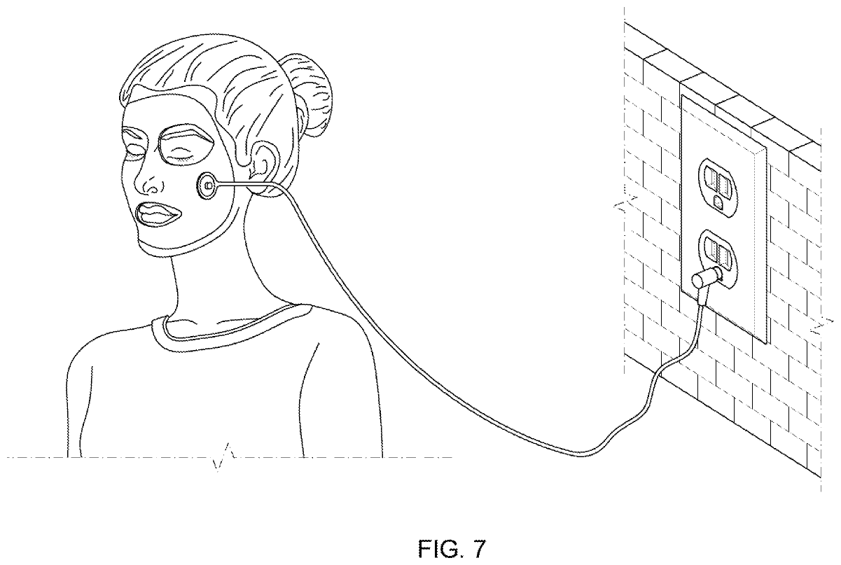

FIG. 8 discloses final view of perspective use as per preferred embodiments of the invention.

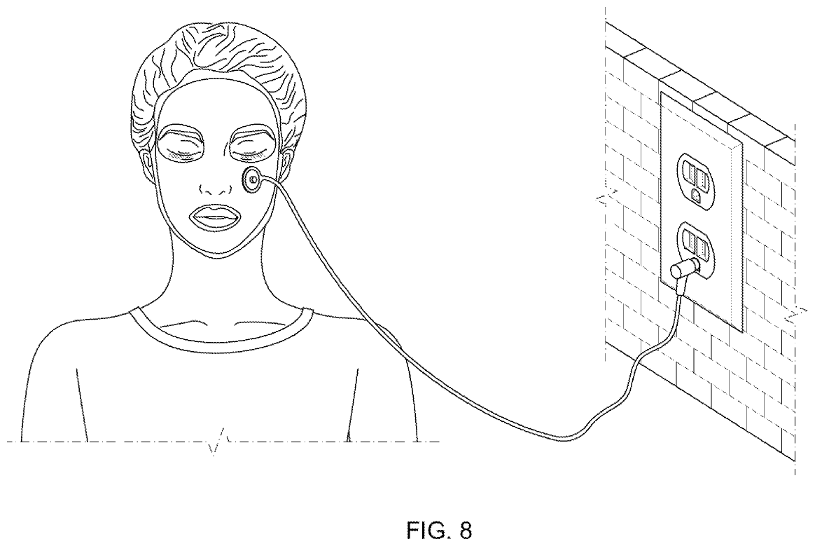

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a conductive skin care cream or jelly which can be applied on part of body coupled with a Grounding Acupressure Device for enhanced treatment.

Typically, Acupressure has been widely used as a primary therapeutic technique due to its minimal side-effects and therapeutic efficacy. However, under conditions of acupressure that may affect the efficacy of acupressure therapy, the grounded response to acupressure stimulation has not been explicitly studied. To achieve the synergistic effect, both strategies are used together in new worlds. This technique is creating a revolution in acupressure research. You know that small e-stim tools can be used by your acupuncturist to inject electrons into your body. With excessive load, we can target very precise areas that are as small as scars that can cause problems or harm to a specific part of the body.

As per another embodiment of the invention the proposed invention provides a method for treating a tissue site comprising delivering energy to the tissue site using a jelly like substance allowing to deliver enhanced mechanical force to the tissue site; producing a thermal adhesion or lesion at the tissue site and remodeling at least a portion of tissue at the tissue site. In related embodiments, the delivery of force can be vectored responsive to one of an energy delivery parameter, a tissue property or patient feedback.

The present invention having the configuration as described above, along the skin care cream or jelly, the acupressure or grounding rod is in close contact with each part of the body desired by the user to perform a rolling (rolling) movement to increase the acupressure and acupressure effect. Any part of the body including but not limited to stomach, back, arms, etc. can be massaged or massaged at the same time smoothly and economically by the use of proposed device and associated gel.

The assembly as per its preferred embodiments cream defines where the body's voltage exits, and the earth's ions enter. The customer can choose precisely where this exchange takes place (entire face, neck, legs and so on.

Furthermore, the Grounding has been demonstrated to increase microcirculation in the face. This helps absorption and delivery of the ingredients to the cells.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: A conductive skin care topical coupled with grounding mechanism comprising:
A grounding wire connected to conductive skin care lotion, gel, crème, etc. to enhance the outcomes of the lotion, gel, crème, etc.
The other side of the grounding wire connected to the grounding port in an electrical outlet;
A conductive sin care tropical in the form of crème, gel, hydrogel patch etc. which can be applied on desired skin area for enhanced and effective treatment;
the cream defines where the body's voltage exits, and the earth's ions enter. Customer can choose precisely where this exchange takes place including but not limited to entire face, neck, legs and so on.
